(19) United States
(12) Patent Application Publication
(10) Pub. No.: US 2025/0025687 A1
(43) Pub. Date: Jan. 23, 2025

(54) MEDICAL LEAD AND METHOD OF SECURING MEDICAL ELECTRODE LEADS

(71) Applicant: DARK INDUSTRIES, LLC, CINCINNATI, OH (US)

(72) Inventors: Mark R Hoeprich, Springboro, OH (US); Daniel Verrill, Piqua, OH (US)

(73) Assignee: DARK INDUSTRIES, LLC, CINCINNATI, OH (US)

(21) Appl. No.: 18/780,369

(22) Filed: Jul. 22, 2024

Related U.S. Application Data
(60) Provisional application No. 63/514,918, filed on Jul. 21, 2023.

(51) Int. Cl. A61N 1/05

(52) U.S. Cl. CPC A61N 1/0558

(57) ABSTRACT

An implantable medical lead having a body with a length with a distal end with a tip, and a proximal end. The proximal end includes an opening to a central lumen extending within and along the length of the implantable lead, and the distal end is provided with electrode contact(s) and with aperture(s) adjacent the electrode contacts. The aperture(s) are formed from through an external surface of the distal end and/or tip through a wall of the implantable electrical lead body to the central lumen. The aperture(s) enable supplying biological adhesive material from the central lumen via the aperture(s) to a desired biological location within a patient. A method for securing the implantable medical lead within the desired biological location using a biological adhesive applied through aperture(s) formed in the distal end of the implantable medical lead is provided.

This application claims the benefit of U.S. Patent Application No. 63/514,918, filed on Jul. 21, 2023, the entire content of which is incorporated herein by reference.

### FIELD OF THE INVENTION

This application relates generally to a medical lead, and more specifically to implantable, electrical leads, and a method for securing such medical electrode leads.

### BACKGROUND

Neuromodulation is a multi-billion dollar per year medical industry. Spinal cord stimulation, one form of neuromodulation, is a well-established and validated therapy for many various chronic pain conditions. Within the field of spinal cord stimulation (SCS), there are two main types of spinal cord stimulator electrode configurations available—percutaneous spinal cord stimulator leads and paddle spinal cord stimulator leads.

Percutaneous spinal cord stimulator leads are typically placed via a small incision, generally in the lumbar region, that allows for placement of Tuohy needles into the epidural space. The implantable electrodes are then steered into position within the epidural space of the spine using metal stylets. Once in place, the steering stylets are removed, the Tuohy needles are removed, and the leads are anchored in place to the fascia of the muscle layer using sleeves that are placed over the leads and secured with suture.

Percutaneous leads account for approximately 90% of all spinal cord stimulator leads placed. Their primary advantage is the minimally invasive method by which they are implanted. Their primary disadvantage is the potential risk of electrode migration. Electrode migration occurs when the lead moves out of the desired location and the stimulation therapy no longer provides meaningful pain relief. Current data suggests that the rate of lead migration is 15-30%, making it one of the biggest potential problems with stimulation therapy overall. Lead migration generally occurs within the first several weeks after implantation, prior to the body forming scar tissue around the electrodes, which scar tissue ultimately serves to hold the electrodes in place.

The alternative to percutaneous leads, paddle leads, are placed by neurosurgeons, and/or orthopedic spine surgeons, via a surgery that requires removal of a portion of the lamina and placing the lead directly in the desired location, making it appropriate for patients with difficult anatomy and who have experienced previous lead migration. The primary advantage of paddle leads is lower migration rates, but this advantage is only obtained using a more invasive surgical option.

Current methods for anchoring less invasive percutaneous stimulator leads are limited. Because the incision is made at a significant distance away from the distal tips of the leads, there are no currently available methods for directly anchoring percutaneous leads within the intended tissue or the epidural space. Presently available methods are limited to various “anchors” that are placed over the leads and secured to the paraspinal fascia with nonabsorbable suture. Since these anchoring devices may be positioned anywhere from 30 to 60 cm away from the distal end or tips of the leads, they are limited in effectiveness, particularly with regard to limiting lead migration of the active distal electrodes on the lead.

### SUMMARY

This application provides a novel type of a percutaneous implantable medical lead for overcoming prior problems with lead migration, and discloses a device and techniques for use of the medical lead, which may include one or multiple electrodes. Electrical stimulation may be delivered via the medical lead, carrying one or more electrodes. Typical implantable leads have an externally exposed electrode, electrodes or electrode contacts in a ring or a series of spaced bands or segmented rings, surrounding the distal end of the lead. Leads are generally constructed, for example, by connecting each electrode to a conductor or connecting wire, disposed within a body of the lead. When completed, an electrical signal generated by an electrically connected electrical stimulator, may be transmitted through one or more conductors and connected electrodes of the lead to generate an electrical field within tissue surrounding the implanted lead, thereby providing electrical stimulation to the tissue surrounding the implanted lead. Connecting wires are embedded within the wall or walls of the body of the lead to electrically connect the electrodes or electrode contacts. The lead also typically includes a central lumen through the lead body from a proximal end to the distal end. The lumen has a diameter sufficient for accepting metal steering stylets. The external diameter of leads typically enables insertion of the lead into and through a Touhy needle, which is then used to position the lead at the desired location. Commonly available lead lengths may range in any size, but, for example, from 20 to 60 cm.

The improved medical lead of the present application, and the techniques or methods for its use, provide a hole or a series of fenestrations, or small holes, between the electrode contacts or bands at the distal end of the lead. The hole(s) are adjacent, meaning either above and/or below, the electrode contacts. Hole(s) may also be alternatively or additionally provided anywhere on the distal end of the lead. It should be understood that the hole(s) are provided at locations and diameters of a size sufficient to permit the flow of fixate material or biological tissue adhesive or glue material to be pressed or injected through the hole(s), passing through and out of the central lumen to the desired biological location.

### BRIEF DESCRIPTION OF DRAWINGS

FIG. 1 is a schematic, cut-away, perspective, illustration and descriptions of spinal cord biology for locating the desired positioning of percutaneous electrode leads within the spinal cord.

FIG. 2 is a schematic, cross-sectional view of spinal cord biology showing the use of a Touhy needle during percutaneous electrode lead placement.

FIGS. 3A, 3B, 3C and 3D are fluoroscopic views showing the use of two Touhy needles during the placement of percutaneous electrode leads in the desired location.

FIG. 4 is a schematic diagram of the percutaneous spinal cord stimulator electrode of the present application.

FIG. 5 is an enlarged schematic view of the distal end or tip of the electrode of the present application with biological adhesive glue shown moving out of the electrode lumen through holes.

FIG. 6 is a cross-sectional schematic view of a portion of the electrode of FIG. 4 taken adjacent and between bands of electrode contacts.

FIG. 7 is a cross-sectional schematic view of a portion of the electrode of FIG. 4 taken through and showing the holes into the electrode lumen.

FIG. 8 schematically illustrates a luer lock connector in open position, prior to engagement with a proximal end of the electrode of FIG. 4.

FIG. 9 schematically illustrates the luer lock connector of FIG. 8, in closed position engaged with the proximal end of the electrode, and prior to engagement with the adhesive glue supply.

The details of the present disclosure are set forth in the accompanying drawings and the description below. Other features, objects, and benefits will be apparent from the description and drawings, and from the claims.

### DETAILED DESCRIPTION

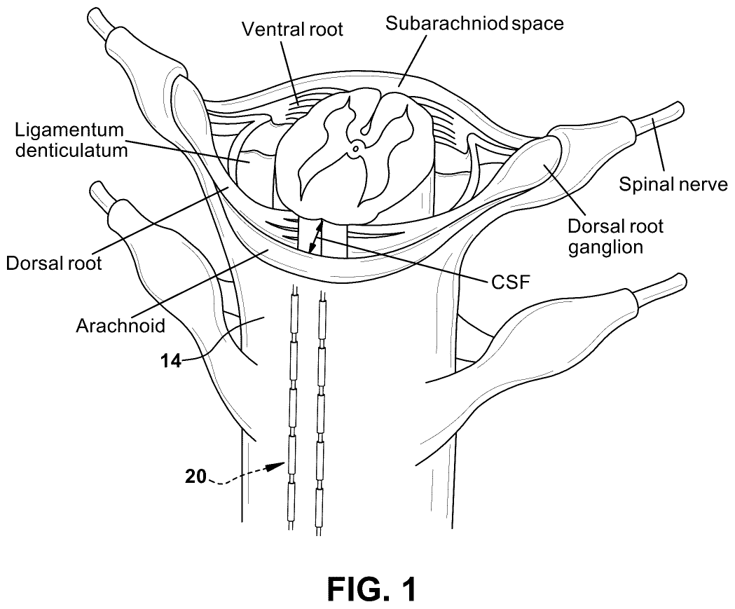

An implantable medical lead device 20, system for use, and techniques for using and securing the medical lead are described herein. In some examples of electrical stimulation therapy, a therapy system includes a medical device 60 configured to generate electrical stimulation signals and a medical lead 20 to deliver or transfer the stimulation signals to the desired tissue T within the patient, as shown schematically and in the images of FIGS. 1, 2 and 3A to 3D. The lead 20 may include one or more electrodes or electrode contacts 22 (e.g., disposed on a longitudinal surface, distal tip, or both of the lead) configured to deliver the electrical stimulation signals to the tissue T. The electrical stimulation energy that may emanate from the electrodes may define an electrical field with respect to the electrodes.

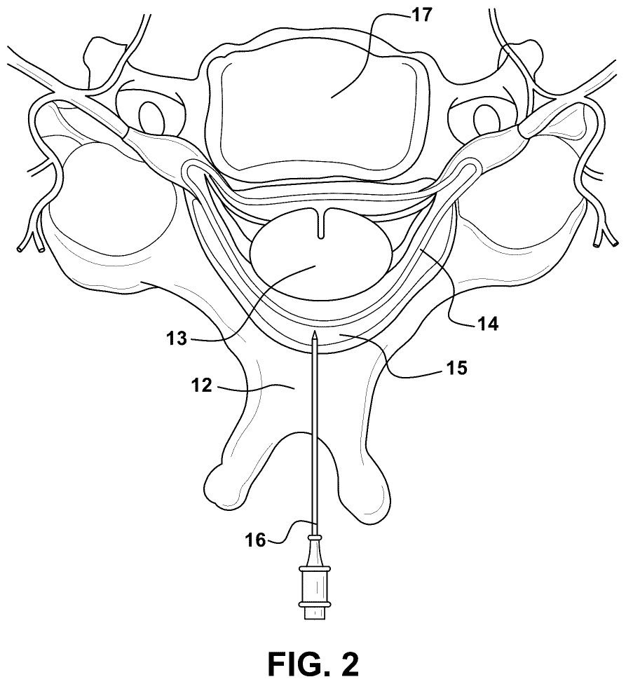

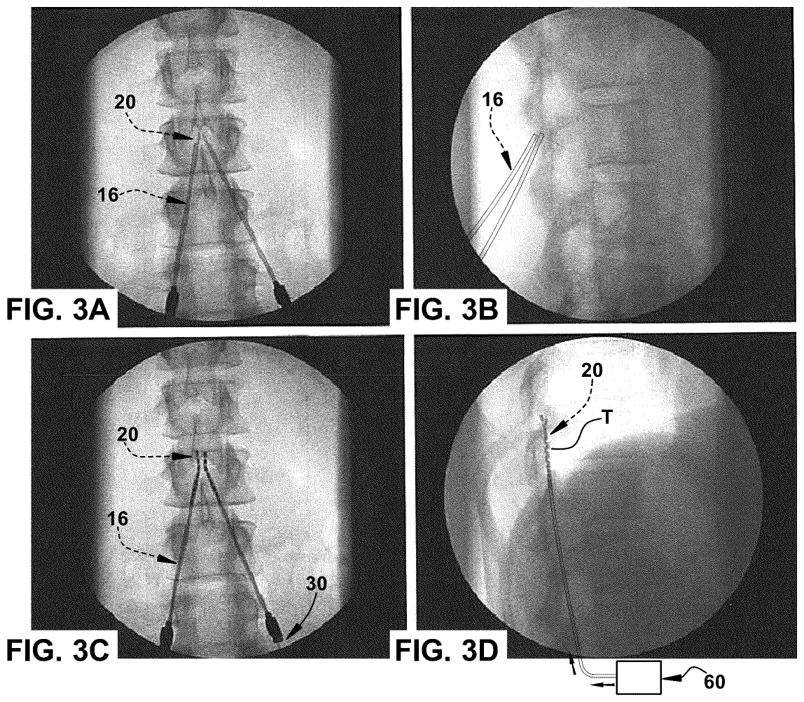

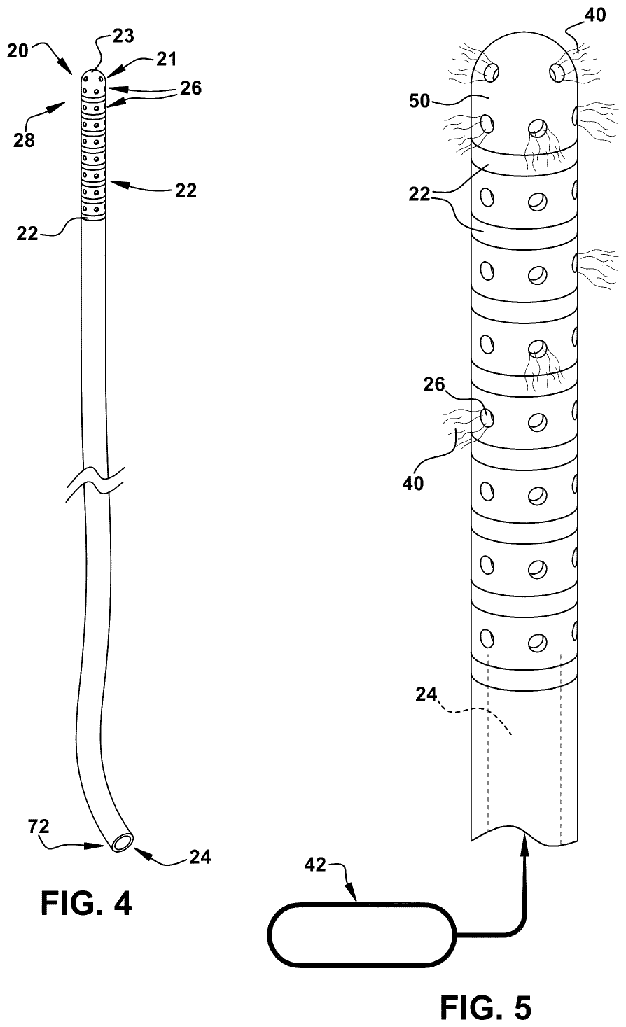

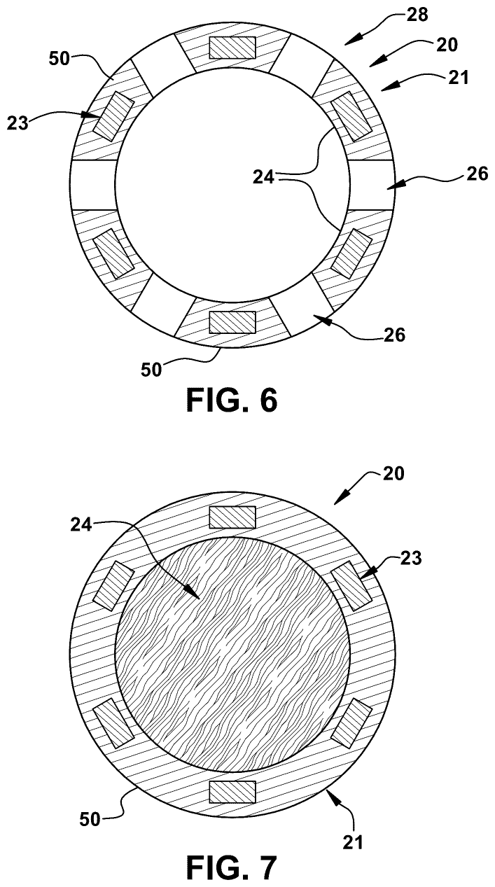

The medical lead 20 of the present disclosure is placed or positioned in the usual fashion for percutaneous leads using a removable metal steering stylet 30 engaged within the central lumen 24 of the lead. Referring to FIGS. 3A-3D, after the lead 20 is positioned in the tissue T at the desired location with respect to the vertebral body 11, dura 14, spinal cord 13 and lamina 12 using the Touhy needle 16, the steering stylet 30 is removed from the central lumen 24. The central lumen 24 is then injected with surgical biological glue material 40 that travels through and along the central lumen, schematically shown in FIG. 5, a portion of which glue material passes out through the distal fenestration(s) 26 to the desired biological location within the tissue T, such as the epidural space 15. The hole(s) 26 within a distal end 28 of a body 21 of the medical lead 20 are positioned adjacent the electrode contact(s) 22, as shown in FIGS. 4 to 7. The hole(s), fenestration(s) or aperture(s) 26, as in FIG. 6, are formed through an external surface 50 of the body 21 and/or a tip 23 of the distal end 28, and enable fluid communication with the central lumen 24, and passage of biological adhesive glue material 40 through hole(s) 26 in the external surface 50 of the body 21 and/or tip 23.

Once the adhesive glue material 40 moves both through the hole(s) 26 formed in the external surface 50 of the body 21 and/or tip 23, as well as into the desired tissue T, the distal end 28 of the medical lead 20 is then stabilized by the adhesive glue material T (after appropriate cure time) in its desired position. This use of a fixate material or biological adhesive glue material 40 maintains the position of the electrode lead 20, giving the patient's body sufficient time to form scar tissue to fixedly secure the electrode within the lead in position. Utilizing surgical biological glue material 40 is a typical method for securing paddle leads, but the use of glue material has not previously been provided with percutaneous leads. Such glue material 40 may remain within the lumen 24 of the lead 20 without impacting the electrical signals supplied to the lead via the electrodes 22. Once the percutaneous medical lead 20 is placed in the desired location, and the glue material 40 is applied, the lead 20 may then be attached to the electrical stimulator or implantable pulse generator 60 for therapy, as would be typical for both percutaneous and paddle leads.

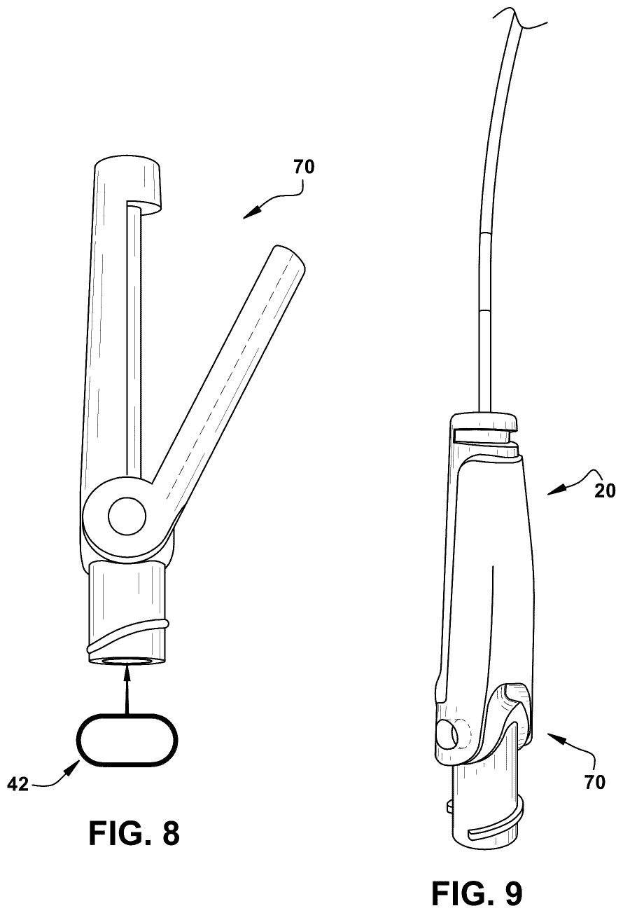

The application of the biological glue 40 may be accomplished using, for example, a removable luer lock connector 70 and glue material supply 42, as in FIG. 8. Once the lead 20 is steered to the desired location through the Touhy needle 16 using the removable stylets 30, the stylets are removed, and the luer lock connector 70 is temporarily attached to a proximal end 72 of the electrode lead, as in FIG. 9. The desired biological adhesive or glue material 40 may then be injected or forced into the central lumen 24 via a conventional syringe or other supply line 42 attached to the luer lock connector 70 spaced from the proximal end 72 of the lead 20. Once the syringe or supply line 42 is used to provide the desired adhesive or glue material 40 into the novel electrode lead 20, the syringe or supply line 42 and luer lock connector 70 may be removed, and the lead is attached to the pulse generator or electrical stimulator 60 as mentioned.

The present novel technique or method for stabilizing the distal end 28 of a percutaneous lead 20 directly within the desired tissue T or epidural space 15 decreases the potential risk of lead migration. As a result, the risk of inadequate spinal cord or other stimulation therapy and the need for revision surgery to correct lead migration is reduced. There is also a reduction in the need for explantation due to lack of adequate therapy. The present solution enables the novel combination of the stability advantage provided by the application of conventional or later developed biological adhesive glue materials 40, together with the advantage of the minimally invasive implantation techniques.

Various examples have been described. These and other examples are within the scope of the following claims.

We claim:

1. An implantable electrical medical lead having a length with a distal end having a body with a tip and a proximal end, wherein the proximal end includes an opening to a central lumen extending within and along the length of the implantable electrical medical lead, wherein the distal end is provided with an electrode contact and with at least one aperture adjacent the electrode contact, and wherein the at least one aperture is formed through an external surface of the body or tip of the distal end into fluid communication with the central lumen.

2. The implantable electrical lead of claim 1, wherein the distal end includes two or more electrode contacts surrounding the external surface of the distal end, and two or more apertures are formed adjacent at least one electrode contact.

3. The implantable electrical lead of claim 1, wherein the distal end includes two or more electrode contacts surrounding the external surface of the body of the distal end, and two or more apertures are formed adjacent at least one of the electrode contacts and/or at the tip of the distal end.

4. A method for securing an implantable medical electrical lead comprising the steps of:
positioning the implantable medical electrical lead within a desired biological location;
forcing a biological adhesive material supplied to and through a central lumen formed within and along an internal length of the implantable medical electrical lead; and
depositing the biological adhesive material from the central lumen through at least one hole formed through a body of the implantable medical electrical lead at a distal end of the implantable medical electrical lead.

5. The method of claim 4, wherein prior to the step of positioning the implantable medical electrical lead forcing the biological adhesive material supplied to and through the central lumen formed within and along the internal length of the implantable medical electrical lead, the following further step is provided:
attaching and securing a luer lock connector into fluid communication with the central lumen engaged at a proximal end of the implantable electrical lead.

6. The method of claim 5, comprising the following further step of:
supplying the central lumen at the proximal end of the implantable electrical lead with biological adhesive material via the luer lock connector from a supply of biological adhesive material.

7. A system for use with the method of any of claims 4 through 6, the system comprising:
an implantable electrical medical lead having a length with a distal end having a body with a tip and a proximal end,
wherein the proximal end includes an opening to a central lumen extending within and along the length of the implantable electrical medical lead,
wherein the distal end is provided with an electrode contact and with at least one aperture adjacent the electrode contact, and
wherein the at least one aperture is formed through an external surface of the body or tip of the distal end into fluid communication with the central lumen enabling passage of biological adhesive material therethrough.

8. The system of claim 7, wherein the distal end includes two or more electrode contacts surrounding the external surface of the distal end, and two or more apertures are formed adjacent at least one electrode contact.

9. The system of claim 8, wherein the distal end includes two or more electrode contacts surrounding the external surface of the body of the distal end, and two or more apertures are formed adjacent at least one of the electrode contacts and/or at the tip of the distal end.
